(19) United States
(12) Patent Application Publication
(10) Pub. No.: US 2026/0182885 A1
(43) Pub. Date: Jul. 2, 2026

(54) PORTABLE BIO-SIGNAL MEASURING DEVICE INCLUDING PEN-SHAPED MAIN BODY

(71) Applicant: SEOUL NATIONAL UNIVERSITY HOSPITAL, Seoul (KR)

(72) Inventors: Joonghee KIM, Seongnam-si (KR); Youngjin CHO, Seongnam-si (KR); Yeonyee E. YOON, Seongnam-si (KR); Jiesuck PARK, Seongnam-si (KR)

(21) Appl. No.: 19/507,646

(22) PCT Filed: Aug. 2, 2024
(86) PCT No.: PCT/KR2024/011438

§ 371 Date: Mar. 21, 2026

(30) Foreign Application Priority Data
Aug. 4, 2023 (KR) 10-2023-0102168

(51) Int. Cl. A61B 5/28; A61B 5/00; A61B 5/021; A61B 5/024; A61B 5/251; A61B 5/352

(52) U.S. Cl. CPC A61B 5/28; A61B 5/02125; A61B 5/02427; A61B 5/251; A61B 5/352; A61B 5/6843; A61B 5/7264; A61B 5/741; A61B 2560/0418

(57) ABSTRACT

Disclosed is a portable bio-signal measuring device including a pen-shaped main body, according to an embodiment of the present invention. The portable bio-signal measuring device including a pen-shaped main body, according to an embodiment of the present invention, comprises: a first electrode which is located in a first region of the main body and measures a first potential of a subject when in contact with a first body part of the subject; a second electrode which is located in a second region of the main body and measures a second potential of the subject when in contact with a second body part of the subject; and one or more processors for calculating an electrocardiogram signal of the subject on the basis of the first potential and the second potential.

### TECHNICAL FIELD

The disclosed embodiments relate to a bio-signal measuring device including a pen-shaped main body.

### CROSS-REFERENCE TO RELATED APPLICATIONS

This application claims priority to Korean Provisional Patent Application No. 10-2023-0102168 filed on Aug. 4, 2023, the content of which is incorporated herein by reference in its entirety.

### BACKGROUND ART

Generally, medical electrocardiograms used in hospitals use 12 channels, of which 6 are limb leads and the other 6 are chest leads. Limb lead electrocardiograms are measured using the extremities of the upper and lower limbs, and chest leads electrocardiograms are measured by attaching electrodes to the chest skin. Therefore, medical 12-channel electrocardiographs measure electrocardiograms by connecting electrodes to the upper and lower limbs and chest using multiple wires.

Recently, simplified devices designed as personal medical devices or in wearable forms for electrocardiogram measurement have been increasingly used. While these devices are convenient to carry, they have limitations in that electrocardiogram measurement is possible only through limb lead measurement or within a very limited area on the chest. The electrocardiogram measured in this manner fails to record comprehensive information of the heart and may only evaluate partial information, and even this is evaluated in a distorted manner, resulting in serious limitations. In particular, most electrocardiogram artificial intelligence systems are trained through data obtained from medical 12-channel electrocardiograms, creating problems in that measurement data obtained from devices having such limitations is not applicable.

Furthermore, conventional electrocardiography devices have limitations in measuring pulse wave and blood pressure information together with electrocardiogram signals (especially in a synchronized manner). This information is as important as electrocardiograms for evaluating a patient's overall hemodynamics, and so measuring the information together is very useful, and integrating the information using artificial intelligence can provide very powerful health assessment.

### DISCLOSURE

### Technical Problem

Embodiments disclosed herein are intended to provide a bio-signal measuring device that enhances user convenience and friendliness by using a portable bio-signal measuring device including a pen-shaped main body.

Embodiments disclosed herein are for predicting estimated blood pressure using an electrocardiogram signal and photoplethysmography (PPG) measured synchronously in a portable bio-signal measuring device including a pen-shaped main body.

### Technical Solution

A portable bio-signal measuring device including a pen-shaped main body according to one embodiment includes a first electrode which is located in a first region of the main body and measures a first potential of a subject when in contact with a first body part of the subject; a second electrode which is located in a second region of the main body and measures a second potential of the subject when in contact with a second body part of the subject; and one or more processors for calculating an electrocardiogram signal of the subject on the basis of the first potential and the second potential.

The first region may be located at one end of the main body, and the second region may be located at the other end of the main body.

The first body part may be located on the subject's chest, and the second body part may be located on at least one body part of the subject's extremities.

The main body may further include a photoplethysmogram (PPG) sensor which is located in a third region of the main body and measures a PPG signal of the subject when in contact with the second body part of the subject.

The third region may be located around the second region such that the second body part is in contact with the PPG sensor simultaneously with the second electrode.

The main body may further include a third electrode provided in a fourth region located between one end of the main body and the other end thereof, the third electrode may be configured to measure a third potential of the subject when located in the fourth region and in contact with a fourth body part of the subject, and the one or more processors may perform a linear combination of the first potential, the second potential, and the third potential to calculate an electrocardiogram signal.

The main body may further include: a rotatable reel; a spring member configured to elastically restore the reel when the reel rotates; and a cable having one end connected to the reel and the other end connected to a fourth electrode, the cable being wound around the reel, and the fourth electrode may be embedded in a fifth region inside the main body, and when the fourth electrode is pulled by an external force, the cable wound around the reel may be unwound and withdrawn to an outside of the main body.

At least one of the second electrode and the PPG sensor may be electrically connected to the portable bio-signal measuring device including the pen-shaped main body, and when an external force is provided to the fourth electrode and the cable is pulled out to an outside of the main body, the electrical connection may be deactivated.

The main body may further include a pressure sensor disposed near at least one region among the first region to the fourth region and configured to detect contact between a subject and at least one of the first electrode, the second electrode, the PPG sensor, and the third electrode.

The main body may further include a speaker that provides vocal guidance for a plurality of first specific points within the first body part that must be contacted sequentially with the first electrode based on the number of contacts detected by the pressure sensor.

The main body may include an indicator in which at least one of an emission pattern, emission intensity, and emission color of an internal light source is controlled by the one or more processors based on an electrical signal generated in response to the number of contacts detected by the pressure sensor.

The one or more processors may: receive a first potential for each of specific points detected by the first electrode sequentially contacting the specific points within the first body part of the subject; receive a second potential detected by the second electrode contacting the second body part of the subject; calculate a electrocardiographic signal for each lead on the basis of the first potential and the second potential at the specific points; generate an asynchronous electrocardiographic array of the subject for each lead on the basis of electrocardiographic signal for each lead; and input input data generated by integrating the electrocardiographic array for each lead into a pre-trained model and output a cardiac health result corresponding to the input data.

The cardiac health result may include at least one of abnormal cardiac rhythm, cardiac disease, abnormal cardiac function, and cardiac health status.

The one or more processors may: receive a first potential for each of specific points detected by the first electrode sequentially contacting the specific points within the first body part of the subject; receive a second potential detected by the second electrode contacting the second body part of the subject; calculate a electrocardiographic signal for each lead on the basis of the first potential and the second potential at the specific points; generate an asynchronous electrocardiographic array of the subject for each lead on the basis of electrocardiographic signal for each lead; generate a PPG array based on a PPG signal detected by the PPG sensor in contact with the second body part, the PPG signal corresponding to the second potential; synchronously map the electrocardiographic array and the PPG array to generate an array pair; and analyze a relationship between the electrocardiographic array and the PPG array included in the array pair and output a health result of the subject.

The one or more processors may calculate a pulse transit time (PTT) on the basis of a difference between a first time point corresponding to a specific waveform of an electrocardiographic signal corresponding to the electrocardiographic array and a second time point corresponding to a specific waveform of a PPG signal corresponding to the PPG array.

The first time point may include a time point corresponding to an R peak of the electrocardiogram signal, and the second time point may include a time point corresponding to a pulse waveform start point of the PPG signal.

The one or more processors may calculate a relationship between the PTT and an estimated blood pressure of the subject using a pre-trained regression model.

The one or more processors may output the estimated blood pressure of the subject as the health result.

### Advantageous Effects

The disclosed embodiments can freely measure limb lead and chest lead electrocardiograms through a single pen-shaped main body, thereby providing a function to measure a 12-lead electrocardiogram with only a small portable device.

The disclosed embodiments can provide a patient with an electrocardiogram measurement environment without undressing, since a thin pen-shaped main body can be inserted between the buttons of a subject's upper garment to measure an electrocardiogram.

The disclosed embodiments strategically place electrodes for bio-signal measurement on the main body, thereby enabling a subject to maintain a correct posture during electrocardiogram measurement through gripping.

The disclosed embodiments can place an electrode for an electrocardiographic signal and a sensor for a photoplethysmographic (PPG) signal in close proximity in a pen-shaped bio-signal measuring device, thereby measuring the two signals synchronously

The disclosed embodiments can measure a blood pressure signal of a subject without a separate blood pressure measuring device by comparing specific points of synchronously measured electrocardiogram signals and photoplethysmogram signals.

### DESCRIPTION OF DRAWINGS

FIG. 1 is a plan view for explaining a portable bio-signal measuring device including a pen-shaped main body according to one embodiment.

FIGS. 2A to 2D are exemplary diagrams for explaining the shape of a portable bio-signal measuring device including a pen-shaped main body according to one embodiment.

FIG. 3 is a plan view for explaining a portable bio-signal measuring device including a pen-shaped main body according to one embodiment.

FIG. 4 is a block diagram for explaining a portable bio-signal measuring device including a pen-shaped main body according to an embodiment.

FIG. 5 is an exemplary diagram for explaining a process of estimating blood pressure by a portable bio-signal measuring device including a pen-shaped main body according to one embodiment.

### MODES OF THE INVENTION

Hereinafter, specific modes of an embodiment will be described with reference to the drawings. The following detailed description is provided to assist a comprehensive understanding of the sensor described in this specification. However, this is merely an example, and the present invention is not limited thereto.

In describing the embodiments, when it is determined that a detailed description of known technology related to the present invention may unnecessarily obscure the gist of the embodiments, the detailed description will be omitted. Furthermore, numbers used in the description process of the embodiments (e.g., first, second, etc.) are merely identification symbols for distinguishing one component from another component.

In the description process of the embodiments, when a member is the to be positioned “on,” “above,” “at an upper end of,” “under,” “below,” “at an lower end of” another member, this includes not only a case where a member is in contact with the other member, but also a case where another member is present between the two members.

In addition, the embodiments described herein may have aspects that are entirely hardware, partially hardware and partially software, or entirely software. The term “unit,” “apparatus,” “module,” “device,” “server,” or “system,” used herein, refers to a computer-related entity such as hardware, a combination of hardware and software, or software. For example, a unit, apparatus, module, device, server, or system may refer to hardware constituting part or all of a platform and/or software such as an application for driving the hardware. As a specific example, the unit, apparatus, module, device, server, or system may be implemented by a processor.

The terminology used herein include terms used to appropriately express preferred embodiments of the present invention, and may vary depending on the intention of the user or operator, or the practices of the field to which the present invention pertains. Therefore, the definition of these terms should be determined based on the contents throughout the present specification. The terms used in the detailed description are merely for describing embodiments and should not be limiting in any way. Unless clearly used otherwise, singular expressions include plural meanings. In this description, expressions such as “comprising” or “having” are intended to designate certain components, numbers, steps, operations, elements, parts thereof, or combinations thereof, and should not be interpreted to exclude the presence or possibility of one or more other components, numbers, steps, operations, elements, parts thereof, or combinations thereof other than those described.

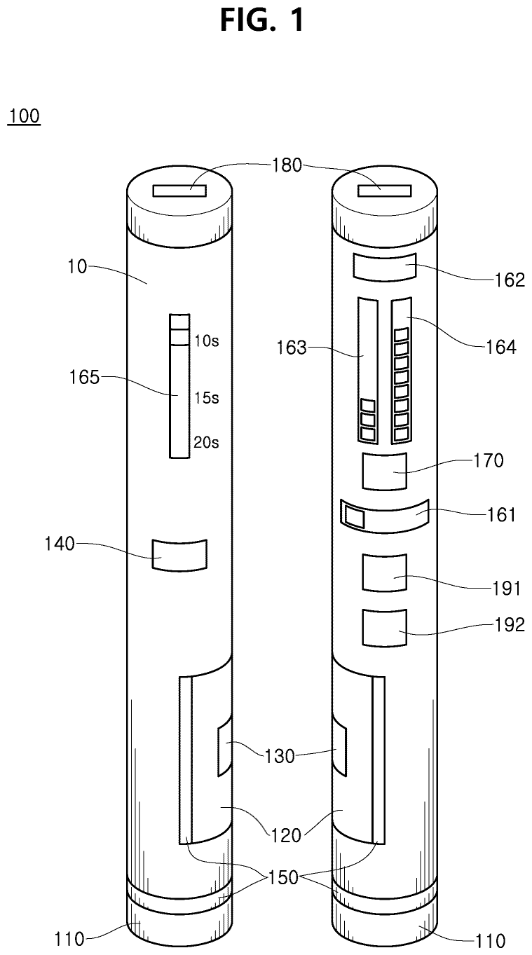

FIG. 1 is a plan view for explaining a portable bio-signal measuring device 100 including a pen-shaped main body according to one embodiment.

Referring to FIG. 1, the portable bio-signal measuring device 100 including the pen-shaped main body includes a main body 10, a first electrode 110, a second electrode 120, and a processor (not shown).

The main body 10 may have a shape of a column formed to a predetermined length with a thin thickness. For example, the main body 10 may have a pen shape or a stamp shape. The main body 10 may preferably have a thickness such that the main body may be inserted between the gaps in the clothing of the subject, and a length such that the main body may be gripped even when inserted between the gaps in the clothing.

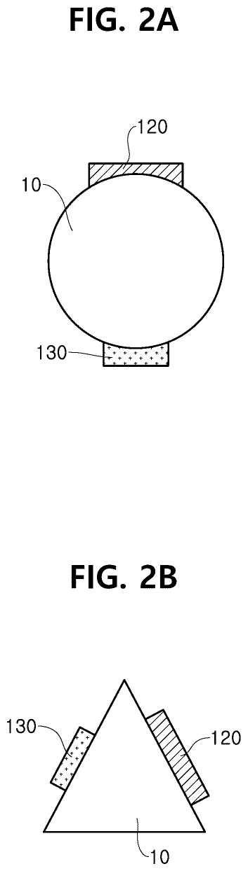

The specific structure and shape of the main body 10 will be described later with reference to FIGS. 2A to 2D.

The first electrode 110 is an electrode which is located in a first region of the main body 10 and measures a first potential of a subject when in contact with a first body part of the subject.

The second electrode 120 is an electrode which is located in a second region of the main body 10 and measures a second potential of the subject when in contact with a second body part of the subject.

The first region may be located at one end of the main body 10, and the second region may be located at another end of the main body 10.

The first region may be located at a very end of the main body 10. The first region may be located at the very end of the main body 10 so that the first electrode 110 may be in contact with the subject's skin when the main body 10 is inserted between gaps in the clothing worn by the subject.

The second region may be located at the other end of the main body 10 positioned in the opposite direction from the one end. The second region may be located at the other end of the main body 10 so that a second body part of the subject gripping the main body 10 may be in contact with the second electrode 120.

A processor (not shown) may calculate an electrocardiogram signal based on the first potential and the second potential. At this time, the electrocardiogram signal may be calculated in voltage units.

Here, the first body part may be located within the subject's chest, and the second body part may be located within any one body part of the subject's extremities.

In particular, the first body region may include a plurality of first specific points in the chest to calculate an electrocardiogram signal for each lead. The first body region may include medically defined body regions to measure a potential value on leads V1, V2, V3, V4, V5, and V6.

For example, the plurality of first specific points may include a first point located near a fourth right rib as a body region in the chest that must be contacted to measure a potential on lead V1. The plurality of first specific points may include a second point located near a fourth left rib as a body region in the chest that must be contacted to measure a potential on lead V2. The plurality of first specific points may include a third point located near the left of the fourth left rib as a body region in the chest that must be contacted to measure a potential on lead V3. At this time, the third point may be located in the left relative to the second point. The plurality of first specific points may include a fourth point located near a fifth right rib as a body region in the chest that must be contacted to measure a potential on lead V4. The plurality of first specific points may include a fifth point located near the left of the fifth right rib as a body region in the chest that must be contacted to measure a potential on lead V5. The plurality of first specific points may include a sixth point located near the middle of the fifth right rib as a body region in the chest that must be contacted to measure a potential on lead V6.

The second body region may include a plurality of second specific points within the extremities to calculate leads V1 to V6. The second body region may include a medically designated body region to measure the potential values required for the calculation of leads V1, V2, V3, V4, V5, and V6. For example, the second specific point may be located on at least one of a left arm, a right arm, a left leg, and a right leg.

In other words, the portable bio-signal measuring device 100 including the pen-shaped main body according to one embodiment may calculate leads V1 to V6 from a combination of the first and second body regions in contact with the first and second electrodes 120, respectively.

According to another embodiment, the first body region and the second body region may include body regions that must be contacted in order to calculate leads I, II, III, aVR, aVL and aVF.

For example, the first and second body regions may include points located on a left arm and a right arm, respectively, as body regions within the extremities to be contacted to measure the potential of lead I. The first and second body regions may include points located on a left leg and a right arm, respectively, as body regions within the extremities to be contacted to measure the potential of lead II. The first and second body regions may include points located on a left leg and a left arm, respectively, as body regions within the extremities to be contacted to measure the potential of lead III.

Referring to FIG. 1, a portable bio-signal measuring device 100 including a pen-shaped main body may further include a photoplethysmographic (PPG) sensor 130.

Here, the PPG sensor 130 is positioned in a third region and may measure a PPG signal of a subject when in contact with a second body part of the subject.

At this time, the third region may be positioned around the second region such that the PPG sensor 130 is in contact with the second body part simultaneously with the second electrode 120. For example, the third region may include a region including a remaining area of the second body part of the subject excluding the second region.

Referring again to FIG. 1, the portable bio-signal measuring device 100 including the pen-shaped main body may further include a third electrode 140.

The third electrode 140 may be an electrode which is located in the fourth region and measures a third potential of the subject when in contact with a fourth body part of the subject.

The fourth region may be located between the first region and the second region of the main body 10. In other words, the fourth region may be located between both ends of the main body 10.

The fourth body part may be a body part on an opposite side of the second body part. For example, when a right hand as the second body part is in contact with the second electrode 120, the fourth body part may be a body part including a left hand.

At this time, a processor (not shown) may calculate an electrocardiogram signal for each lead through a linear combination of respective potentials (a first potential, a second potential, a third potential) measured at the first electrode 110, the second electrode 120, and the third electrode 140.

The processor (not shown) may calculate at least one of leads aVR, aVL, and aVF using the first to third potentials measured by varying the combinations of body parts in contacted with the first electrode 110, the second electrode 120, and the third electrode 140.

For example, the first electrode 110 may be in contact with a left leg, the second electrode 120 may be in contact with a right hand, and the third electrode 140 may be in contact with a left hand. Through this, two of leads I, II, and III may be measured in a synchronized manner, and the remaining one and aVR, aVL, and aVF may be calculated through their linear combination.

Referring to FIG. 1, the portable bio-signal measuring device 100 including the pen-shaped main body may further include a pressure sensor 150.

The pressure sensor 150 may be a sensor which is disposed near at least one region among the first region to the fourth region and detects contact between a subject and at least one of the first electrode 110, the second electrode 120, the PPG sensor 130, and the third electrode 140.

The pressure sensor 150 may measure pressure applied to the main body 10 when at least one of the first electrode 110, the second electrode 120, the PPG sensor 130, and the third electrode 140 is in contact with the subject's skin, based on the action-reaction principle.

For precise measurement, the pressure sensor 150 may preferably be disposed in the first region to the fourth region where the first electrode 110, the second electrode 120, the PPG sensor 130, and the third electrode 140 are disposed.

Meanwhile, the pressure sensor 150 has been described as being located in the vicinity of the first electrode 110, the second electrode 120, the PPG sensor 130, and the third electrode 140, but this is exemplary, and without limitation to the type of measuring device, the pressure sensor is intended to be located in the vicinity of the measuring device with which the subject is in contact.

Referring to FIG. 1, the portable bio-signal measuring device 100 including the pen-shaped main body may further include a plurality of indicators.

The plurality of indicators may control an operating state of a light source to visually display real-time information or instructions to a user. The indicators may control operation of the light source to guide a current contact sequence, step, state, and the like.

The user may understand the contact sequence, step state, and the like associated with the operation of the light source and be guided by visual instructions of the indicators.

Specifically, the first indicator 161 may guide contact with a next body part by changing an emission pattern, intensity, color, and the like of the light source.

At this time, the first indicator 161 may change an emission state according to an electrical signal generated based on a number of contacts detected by the pressure sensor 150.

For example, the first indicator 161 may determine that the user has followed a visual instruction as the number of contacts detected by the pressure sensor 150 increases, and may guide the next visual instruction.

On the other hand, when the number of contacts does not increase, the first indicator 161 may re-guide to follow the current visual instruction. For example, the first indicator 161 may re-implement the emission state of the light source corresponding to the current visual instruction.

The second indicator 162 may guide the state of the portable bio-signal measuring device 100 including the pen-shaped main body. For example, the second indicator 162 may guide, through the color of the light source, whether the portable bio-signal measuring device 100 including the pen-shaped main body is currently in use or whether there is poor contact.

In particular, the second indicator 162 may guide the bio-signal currently being measured by the plurality of electrodes. In other words, the second indicator 162 may guide the user to identify whether the bio-signal currently being measured is an electrocardiogram signal or a PPG signal.

A 3-1 indicator 163 may indicate the remaining amount of the components processing the electrocardiogram signal. A 3-2 indicator 164 may indicate the remaining amount of components processing the PPG wave.

Referring to FIG. 1, the portable bio-signal measuring device 100 including the pen-shaped main body may further include a measurement time setting portion 165.

The measuring time setting portion 165 is capable of controlling a minimum unit duration that an electrode of the portable bio-signal measuring device 100 including the pen-shaped main body must remain in contact with a subject in order to measure a bio-signal.

For example, the measuring time setting portion 165 may adjust the time that the electrode must contact the subject to measure a bio-signal to any one unit of 10 seconds, 15 seconds, or 20 seconds.

At this time, when a set minimum unit has not elapsed after a contact time point detected from a pressure sensor, the first indicator 161 and the second indicator 162 may maintain a current light emission state of a light source to cause a user to follow current visual instructions again,.

Referring to FIG. 1, the portable bio-signal measuring device 100 including the pen-shaped main body may further include a speaker 170.

The speaker 170 may guide to a next body part to be contacted with the subject based on the number of contacts with the subject detected by the pressure sensor 150.

Specifically, the speaker 170 may provide voice guidance to a plurality of first specific points within a first body region to be successively contacted by the first electrode 110 based on the number of contacts with the subject detected by the pressure sensor 150.

For example, when measuring an electrocardiogram, when the pressure sensor 150 has zero contacts with the subject, the speaker 170 may, in a first order, guide the user to a first specific point where the first electrode 110 and/or the second electrode 120 must contact in order to measure a V1 lead signal. At this time, the speaker 170 may guide the user to bring the first electrode 110 into contact with a region near to the subject's fourth right rib and the second electrode 120 with an index finger of the subject.

For example, when measuring an electrocardiogram, when the pressure sensor 150 has one contact with the subject, the speaker 170 may, in a second order, guide the user to a first specific point where the first electrode 110 and/or the second electrode 120 must contact in order to measure a V2 lead signal. At this time, the speaker 170 may guide the user to bring the first electrode 110 into contact with a region near the subject's fourth left rib and the second electrode 120 with an index finger of the subject.

Referring to FIG. 1, the portable bio-signal measuring device 100 including the pen-shaped main body may further include a connector 180.

The connector 180 may include a physical connection portion configured to transmit data between different devices and to supply power. The connector 180 may be provided at one end of the main body 10 and may include a data pin for transmitting and receiving data and a power pin for supplying power.

Referring to FIG. 1, the portable bio-signal measuring device 100 including the pen-shaped main body may further include a power supply portion 191.

When the power supply portion 191 is turned on, the power supply portion 191 may electrically connect an external power source and the portable bio-signal measuring device 100 including the pen-shaped main body so that the portable bio-signal measuring device 100 including the pen-shaped main body may initiate operation.

Referring to FIG. 1, the portable bio-signal measuring device 100 including the pen-shaped main body may further include a reset portion 192.

When the reset portion 192 is turned on, the reset portion 192 may transmit a reset signal to initialize settings of the portable bio-signal measuring device 100 including the pen-shaped main body. For example, when the reset portion 192 is turned on, the reset portion 192 may set an indicator to restart a light emission operation from an initial state.

FIGS. 2A to 2D are exemplary diagrams for explaining the shape of a portable bio-signal measuring device 100 including a pen-shaped main body according to one embodiment.

As shown in FIGS. 2A to 2D, the portable bio-signal measuring device 100 including the pen-shaped main body may include the pen-shaped main body 10 formed in a columnar shape having a thin thickness with a predetermined length.

Here, the main body 10 may have a thickness sufficient for a subject to easily grip the main body 10 with fingers. For example, the main body 10 may have a thickness sufficient to be gripped by a thumb and an index finger. As another example, the main body 10 may have a thickness sufficient to grip an electrode formed on a front surface thereof with a palm.

Preferably, when the main body 10 is a cylinder, a cross-section of the main body 10 may have a diameter of about 5 cm or less. When the main body 10 is a polygonal column, a cross-section of the main body 10 may have a width and a height of about 5 cm or less each.

The main body 10 may have a length such that one end and the other end of the main body 10 may comfortably contact a subject's chest and hand, respectively. For example, the length of the main body 10 may include a known average body length between a fist and the chest when a forearm holding the subject's fist is parallel to the chest.

Preferably, the main body 10 may have a length of approximately 12 cm to 20 cm. Meanwhile, the above-described thickness or length of the main body 10 is exemplary and is not limited to a restricted range. For example, the length of the main body 10 may include a short length of less than 12 cm.

Referring again to FIGS. 2A to 2D, the portable bio-signal measuring device 100 including the pen-shaped main body includes a second electrode 120 and a PPG sensor 130.

The second electrode 120 and the PPG sensor 130 may be disposed in a third region located around the second region where the second electrode 120 is positioned, so that the second electrode 120 and the PPG sensor 130 may be simultaneously contacted by one body part, as shown in FIG. 1.

Unlike what was described above in FIG. 1, the second electrode 120 and the PPG sensor 130 may be contacted by different body parts and may be disposed at positions that may be gripped by different body parts.

As shown in FIGS. 2A to 2C, the second electrode 120 and the PPG sensor 130 may be disposed in different regions so that they may be gripped by a thumb and an index finger or by different body parts including a thumb and a middle finger.

For example, the second electrode 120 and the PPG sensor 130 may be disposed on opposing surfaces of the main body 10. As another example, the second electrode 120 and the PPG sensor 130 may be disposed on adjacent surfaces of the main body 10.

As shown in FIG. 2D, the second electrode 120 and the PPG sensor 130 may be disposed such that the second electrode 120 surrounds a front surface of the main body 10 so as to be gripped by a finger and a palm, and the PPG sensor 130 may be disposed on one surface of the main body 10.

Meanwhile, in FIGS. 2A to 2D, the shape of the main body 10 is described as a columnar shape including a cylinder, a triangular column, and a rectangular column, but this is exemplary, and the shape of the main body 10 is not limited thereto.

The above-described shape and structure of the portable bio-signal measuring device 100 including the pen-shaped main body shown in FIGS. 2A to 2D are an exemplary embodiment, and are not limited thereto, and may be implemented in various shapes and structures.

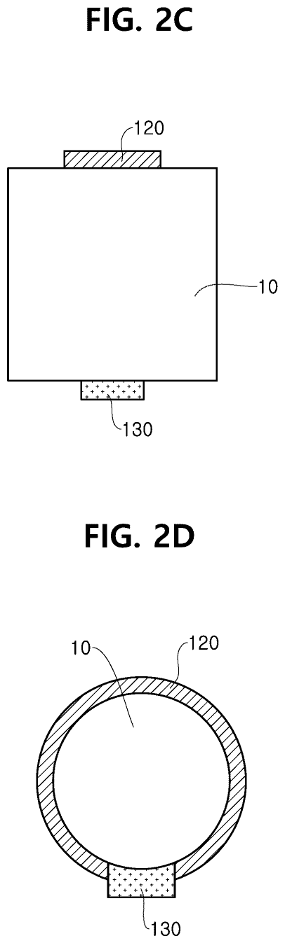

FIG. 3 is a plan view for explaining a portable bio-signal measuring device 100 including a pen-shaped main body according to one embodiment.

Referring to FIG. 3, the portable bio-signal measuring device 100 including the pen-shaped main body according to one embodiment may further include a fourth electrode 350.

The first electrode 310, second electrode 320, PPG sensor 330, and third electrode 340 of FIG. 3 are identical to those of FIG. 1, and for convenience of description, description of overlapping components will be omitted.

The fourth electrode 350 is an electrode for measuring an electrocardiogram signal and may maintain contact with a subject through an adhesive material. Preferably, the fourth electrode 350 is brought into contact with the subject through assistance of another person and may be used for patients who have difficulty in gripping the main body 10.

The fourth electrode 350 may be embedded in a fifth region inside the main body 10, and when pulled by an external force, a cable connected to one end of the fourth electrode 350 may be unwound and withdrawn to an outside of the main body 10.

The cable may be wound on a rotatable reel embedded in the main body 10 with one end connected to the reel and another end connected to the fourth electrode 350. The main body 10 may further include a spring member elastically restored when the reel rotates so that the fourth electrode 350 may be re-embedded in the fifth region by elasticity when the external force is removed.

The fourth electrode 350 is an electrode provided for electrocardiogram measurement in the same manner as the first electrode 110, and whether it is activated may be determined based on whether the person (e.g., user) who grips the main body 10 and the subject (e.g., examinee) to be measured are the same person.

Hereinafter, the case where the user and the subject are the same and the case where they are different are defined as a self-measurement mode and an other-measurement mode, respectively.

Specifically, the fourth electrode 350 is preferably activated in the other-measurement mode. Meanwhile, the second electrode 120 and the PPG sensor 130 are preferably deactivated in the other-measurement mode so that the user's bio-signal is not misconceived as a measurement result of the subject.

On the other hand, in the self-measurement mode, the fourth electrode 350 is deactivated, and the first electrode 110, the second electrode 120, and the PPG sensor 130 are preferably activated.

The self-measurement mode and the other-measurement mode may be determined by a user through input means including buttons, switches, levers, and the like, provided on the main body 10, but such input means are not limited to the above-mentioned examples.

For example, the setting of the self-measurement mode and the other-measurement mode may be triggered by an external force resulting from withdrawal of the fourth electrode 350. The setting of the self-measurement mode and the other-measurement mode may be triggered by input means actuated by an external force.

Specifically, when an external force sufficient to withdraw the cable to an outside of the main body 10 is provided, the fourth electrode 350 may be electrically connected to the portable bio-signal measuring device 100 including the pen-shaped main body. When the external force is provided, an input means connected to the cable may be switched on.

In other words, when an external force sufficient to withdraw the cable to an outside of the main body 10 is provided, at least one of the second electrode 120 and the PPG sensor 130 may deactivate electrical connection with the portable bio-signal measuring device 100 including the pen-shaped main body.

Meanwhile, the term “user” as used herein may refer to an examiner as a person who grips the main body 10. In other words, in a self-measurement mode, the user may mean an examinee as the same person as the subject, but in an other-person measurement mode, the user may mean an examinee as a person other than the subject.

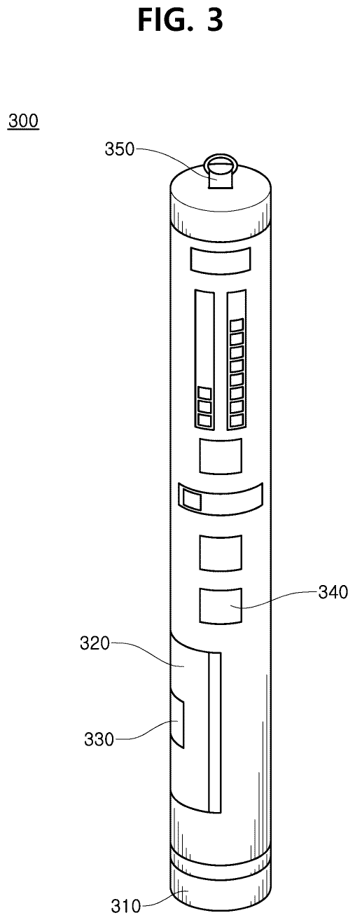

FIG. 4 is a block diagram for explaining a portable bio-signal measuring device 100 including a pen-shaped main body according to an embodiment.

Referring to FIG. 4, the portable bio-signal measuring device 100 including the pen-shaped main body according to one embodiment includes a processor 410 and a memory 420.

Meanwhile, herein, the electrodes, bio-signals, body parts, and the like are the same as those described in FIG. 1, and descriptions of overlapping parts will be omitted.

The portable bio-signal measuring device 100 including the pen-shaped main body according to one embodiment may measure an electrocardiographic signal using a first electrode 110 and a second electrode 120 and analyze the measured electrocardiographic signal to analyze electrocardiographic health.

First, the processor 410 receives a first potential for each specific point detected by the first electrode 110 sequentially contacting specific points within a first body part.

Thereafter, the processor 410 receives a second potential sensed as the second electrode 120 contacts the second body part. By contacting different points within the first body part, the second potential may be measured a plurality of times correspondingly at the same time point whenever the first potential is measured a plurality of times. In other words, the second potential may be measured a corresponding number of times as the first potential so that the second potential may be paired with the first potential.

For example, when the first electrode 110 sequentially contacts first specific points such as a fourth left rib and a fourth right rib to measure the first potential of six leads, the second potential may also be measured six times at the same time points.

The processor 410 may generate an asynchronous electrocardiogram array for each lead of the subject based on the first potential and the second potential.

For example, an asynchronous electrocardiogram array for each lead may mean an electrocardiogram array generated by separating first potentials for each lead that do not occur simultaneously, because the first potential for each lead is obtained by repeatedly bringing the first electrode 110 into contact with different points.

At this time, the electrocardiogram array for each lead may include a vector having a 1*N array. The electrocardiogram array for each lead is converted into an embedding vector processed through an encoder. The encoder may be trained to output a corresponding embedding vector when an electrocardiogram signal for each lead is input. The encoder may be trained to preprocess the electrocardiogram signal for each lead and then output a corresponding embedding vector when the preprocessed electrocardiogram signal for each lead is input. To train such an encoder, training data may be randomly applied with time axis shifts so that the electrocardiogram signals for each lead have asynchronous relationships with each other. The encoder may be trained to output embedding vectors corresponding to the preprocessed electrocardiogram signals for each lead. In this case, the embedding vector is generated by being processed as a single-channel signal and may be an embedding vector extracted for each lead.

Here, N may mean a value calculated by multiplying the time at which the first and second potentials are measured by the frequency of the first and second potentials. Meanwhile, it is assumed that the measurement time and frequency of the synchronously measured first and second potentials are the same with each other.

The processor 410 integrates the electrocardiogram array for each lead to generate input data, inputs the generated input data into a pre-trained model, and outputs a cardiac health result corresponding to the input data.

Here, the input data may include a vector having an L*N array. For example, the input data may include a vector of an L*N array generated by concatenation of electrocardiogram arrays for each lead, and L may represent the number of leads.

In another example, the input data may represent electrocardiogram image data in which electrocardiogram waveforms generated based on the preprocessed electrocardiogram signals for each lead are arranged on a two-dimensional plane in a specific format.

Here, the electrocardiogram signals for each lead may represent data that visually shows asynchronous relationships, as described above, and these are arranged in a predetermined format to create a single image (e.g., 12-lead electrocardiogram, 6-lead electrocardiogram: I, II, III, aVR, aVL, aVF). At this time, an encoder may extract an embedding vector based on the image as input data of the pre-trained model. To train such an encoder, the electrocardiogram signals for each lead that are desynchronized by random shifts along a time axis as described above may be utilized as training data.

Meanwhile, the encoder may be an encoder generated based on at least one of a convolutional neural network, a recurrent neural network, a multilayer perceptron, and a transformer.

The pre-trained model may be a model trained to classify at least one of abnormality of cardiac rhythm, presence of heart disease, and abnormality of cardiac function corresponding to the input data.

As a specific example, the pre-trained model may be trained to classify the presence of at least one of cardiac fibrillation, atrial fibrillation, atrial tachycardia, atrial decompensated fibrillation, ventricular tachycardia, ventricular fibrillation, idioventricular rhythm, paroxysmal atrial tachycardia, junctional ventricular rhythm, atrioventricular block, escape rhythm, and paroxysmal atrial rhythm as an abnormality of cardiac rhythm.

As a specific example, the pre-trained model may be trained to classify at least one of cardiac arrest, respiratory failure, myocardial infarction, myocardial injury, and pulmonary edema as a cardiac disease.

As a specific example, the pre-trained model may be trained to classify at least one of left ventricular failure, right ventricular failure, the presence of cardiac structural changes, and the presence of valvular dysfunction as an abnormality of cardiac function.

As another example, the pre-trained model may be a model trained to predict a health status corresponding to input data.

As a specific example, the pre-trained model may be trained to output at least one prediction value among cardiac health age, cardiac life expectancy, cardiovascular disease occurrence risk, cardiac exercise capacity, and cardiac surgery risk as a health status.

Meanwhile, the pre-trained model may be a learning model including a fully connected layer and/or a dense layer, and may be trained to perform classification and/or regression tasks.

The portable bio-signal measuring device 100 including the pen-shaped main body according to one embodiment may use the first electrode 110 to the PPG sensor 130 to measure not only an electrocardiogram signal but also a PPG signal, and may estimate blood pressure using the electrocardiogram signal and the PPG signal.

First, the processor 410 receives a first potential for each specific point detected when the first electrode 110 sequentially contacts specific points within a first body part.

The processor 410 receives a second potential detected by the second electrode 120 in contact with a second body part.

The processor 410 may generate an asynchronous electrocardiogram array for each lead of the subject based on the first potential and the second potential. The processor 410 generates a PPG array based on a PPG signal detected by the PPG sensor 130 in contact with the second body part.

The PPG signal corresponds to the second potential, and here, correspondence may mean that the second electrode 120 and the PPG sensor 130 are simultaneously brought into contact with the body part so that the signals are measured at the same time. In other words, the PPG signal may be measured synchronously with the second potential through strategic placement of the second electrode 120 and the PPG sensor 130.

Here, the PPG array may include a vector generated based on the PPG signal. The PPG array may include an embedding vector processed through an encoder. In this case, when PPG signal is input, the encoder may output a embedding vector corresponding thereto.

The PPG array may include a vector having an array size of W*N′. Here, W may mean the number of times the PPG signal has been measured corresponding to the second potential, and N′ may be calculated as a product of T′ and F′. At this time, T′ may mean the time during which the PPG signal has been measured, and F′ may mean the frequency of the PPG signal.

The processor 410 generates an array pair by synchronously mapping the electrocardiogram array and the PPG array.

The processor 410 may generate an array pair by mapping an electrocardiographic array and a PPG array generated based on bio-signals measured in the same interval.

For example, the processor 410 may generate an array pair by mapping an electrocardiographic array generated based on a first potential measured between 0 and 15 seconds for a lead V1 measurement and a second potential measured between 0 and 15 seconds for the lead V1 measurement with a PPG array generated based on a PPG signal measured between 0 and 15 seconds.

The processor 410 outputs a health result of the subject by analyzing a relationship between the electrocardiographic array and the PPG array included in the array pair. Specifically, the processor 410 may output an estimated blood pressure as a health result.

The processor 410 may predict an estimated blood pressure of the subject by analyzing a difference of specific time points between the electrocardiographic array and the PPG array included in the array pair. The processor 410 may predict the estimated blood pressure based on a difference between a first time point corresponding to a specific waveform of an electrocardiographic signal corresponding to the electrocardiographic array and a second time point corresponding to a PPG signal corresponding to the PPG array.

In this case, the processor 410 may calculate a pulse transit time (PTT) based on the difference between the first time point and the second time point. The processor 410 can predict an estimated blood pressure of the subject based on the pulse transit time.

The memory 420 stores one or more instructions executed by the processor 410.

The memory 420 may store various data used by the processor 410. For example, the memory 420 may include input data or output data for software (e.g., a program executed by the processor 410 and/or instructions related to the program).

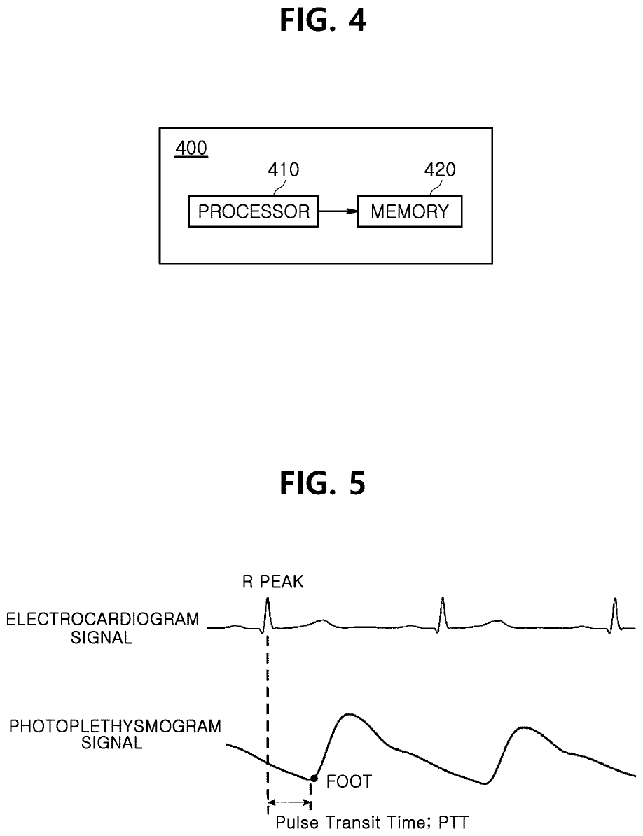

FIG. 5 is an exemplary diagram for explaining a process of estimating blood pressure by a portable bio-signal measuring device 100 including a pen-shaped main body according to one embodiment.

The bio-signal graph at the upper portion is an electrocardiogram signal generated based on the first potential and the second potential. The bio-signal graph at the lower portion is a PPG signal generated based on the PPG signal.

The processor may detect a first point corresponding to an R peak of an electrocardiogram signal corresponding to the electrocardiogram array and detect a second point corresponding to a foot point of a PPG signal corresponding to the PPG array.

The R peak is the highest voltage point of the waveform generated by the electrocardiogram signal. The R peak may mean a point in an electrical activity of the heart when the ventricles begin to contract and spread blood from the ventricles into the body. The foot point is a reference point in the PPG signal, which may indicate the starting point of the PPG wave.

In this case, the processor may calculate the PPT based on the time difference between the first time point corresponding to the R peak in the electrocardiogram signal and the second time point corresponding to the foot point appearing in the PPG signal.

Meanwhile, embodiments of the present invention may include a program for performing the methods described herein on a computer, and a computer-readable recording medium including the program. The computer-readable recording medium may include program instructions, local data files, local data structures, and the like, either individually or in combination. The medium may be those specially designed and configured for the present invention, or those commonly available in the field of computer software. Examples of computer-readable recording media include magnetic media such as hard disks, floppy disks, and magnetic tapes, optical recording media such as CD-ROMs, DVDs, and hardware devices specially configured to store and perform program instructions such as ROMs, RAMs, flash memories, and the like. Examples of the program may include not only machine language code such as that created by a compiler, but also high-level language code that may be executed by a computer using an interpreter or the like.

Representative embodiments of the present invention have been described in detail above, but a person having ordinary skill in the art to which the present invention pertains will understand that various modifications are possible to the aforementioned embodiments within the scope not departing from the scope of the present invention. Therefore, the scope of rights of the present invention should not be limited to the described embodiments, but should be defined not only by the claims described below, but also by those equivalent to these claims.

### Industrial Applicability

A portable bio-signal measuring device including a pen-shaped main body according to one embodiment enables free electrocardiogram measurement through the pen-shaped main body having strategically arranged electrodes and sensors, and enables medical analysis based on the measured bio-signals, and thus can be utilized in medical devices and digital healthcare industries.

1. A portable bio-signal measuring device including a pen-shaped main body, the device comprising:
a first electrode which is located in a first region of the main body and measures a first potential of a subject when in contact with a first body part of the subject;
a second electrode which is located in a second region of the main body and measures a second potential of the subject when in contact with a second body part of the subject; and
one or more processors for calculating an electrocardiogram signal of the subject on the basis of the first potential and the second potential.

2. The device of claim 1, wherein the first region is located at one end of the main body, and the second region is located at the other end of the main body.

3. The device of claim 1, wherein the first body part is located on the subject's chest, and the second body part is located on at least one body part of the subject's extremities.

4. The device of claim 1, wherein the main body further includes a photoplethysmogram (PPG) sensor which is located in a third region of the main body and measures a PPG signal of the subject when in contact with the second body part of the subject.

5. The device of claim 4, wherein the third region is located around the second region such that the second body part is in contact with the PPG sensor simultaneously with the second electrode.

6. The device of claim 4, wherein the main body further includes a third electrode provided in a fourth region located between one end of the main body and the other end thereof,
the third electrode is configured to measure a third potential of the subject when located in the fourth region and in contact with a fourth body part of the subject, and
the one or more processors perform a linear combination of the first potential, the second potential, and the third potential to calculate an electrocardiogram signal.

7. The device of claim 4, wherein the main body further includes:
a rotatable reel;
a spring member configured to elastically restore the reel when the reel rotates; and
a cable having one end connected to the reel and the other end connected to a fourth electrode, the cable being wound around the reel,
wherein the fourth electrode is embedded in a fifth region inside the main body, and when the fourth electrode is pulled by an external force, the cable wound around the reel is unwound and withdrawn to an outside of the main body.

8. The device of claim 4, wherein at least one of the second electrode and the PPG sensor is electrically connected to the portable bio-signal measuring device including the pen-shaped main body, and when an external force is provided to the fourth electrode and the cable is pulled out to an outside of the main body, the electrical connection is deactivated.

9. The device of claim 6, wherein the main body further includes a pressure sensor disposed near at least one region among the first region to the fourth region and configured to detect contact between a subject and at least one of the first electrode, the second electrode, the PPG sensor, and the third electrode.

10. The device of claim 9, wherein the main body further includes a speaker that provides vocal guidance for a plurality of first specific points within the first body part that must be contacted sequentially with the first electrode based on the number of contacts detected by the pressure sensor.

11. The device of claim 10, wherein the main body includes an indicator in which at least one of an emission pattern, emission intensity, and emission color of an internal light source is controlled by the one or more processors based on an electrical signal generated in response to the number of contacts detected by the pressure sensor.

12. The device of claim 1, wherein the one or more processors:
receive a first potential for each of specific points detected by the first electrode sequentially contacting the specific points within the first body part of the subject;
receive a second potential detected by the second electrode contacting the second body part of the subject;
calculate a electrocardiographic signal for each lead on the basis of the first potential and the second potential at the specific points;
generate an asynchronous electrocardiogram array of the subject for each lead on the basis of electrocardiographic signal for each lead; and
input input data generated by integrating the electrocardiogram array for each lead into a pre-trained model and output a cardiac health result corresponding to the input data.

13. The device of claim 12, wherein the cardiac health result includes at least one of abnormal cardiac rhythm, cardiac disease, abnormal cardiac function, and cardiac health status.

14. The device of claim 4, wherein the one or more processors:
receive a first potential for each of specific points detected by the first electrode sequentially contacting the specific points within the first body part of the subject;
receive a second potential detected by the second electrode contacting the second body part of the subject;
calculate a electrocardiographic signal for each lead on the basis of the first potential and the second potential at the specific points;
generate an asynchronous electrocardiogram array of the subject for each lead on the basis of electrocardiographic signal for each lead;
generate a PPG array based on a PPG signal detected by the PPG sensor in contact with the second body part, the PPG signal corresponding to the second potential;
synchronously map the electrocardiographic array and the PPG array to generate an array pair; and
analyze a relationship between the electrocardiographic array and the PPG array included in the array pair and output a health result of the subject.

15. The device of claim 14, wherein the one or more processors calculate a pulse transit time (PTT) on the basis of a difference between a first time point corresponding to a specific waveform of an electrocardiographic signal corresponding to the electrocardiographic array and a second time point corresponding to a specific waveform of a PPG signal corresponding to the PPG array.

16. The device of claim 15, wherein the first time point includes a time point corresponding to an R peak of the electrocardiogram signal, and the second time point includes a time point corresponding to a pulse waveform start point of the PPG signal.

17. The device of claim 15, wherein the one or more processors calculate a relationship between the PTT and an estimated blood pressure of the subject using a pre-trained regression model.

18. The device of claim 14, wherein the one or more processors output the estimated blood pressure of the subject as the health result.
